(19) United States
(12) Patent Application Publication
(10) Pub. No.: US 2025/0143623 A1
(43) Pub. Date: May 8, 2025

(54) METHOD AND SYSTEM OF DETECTING ARRYTHMIA

(71) Applicant: THE UNIVERSITY OF SYDNEY, Sydney, New South Wales (AU)

(72) Inventors: Zihuai LIN, Sydney, New South Wales (AU); Branka VUCETIC, Sydney, New South Wales (AU); Xucun YAN, Sydney, New South Wales (AU)

(73) Assignee: THE UNIVERSITY OF SYDNEY, Sydney, New South Wales (AU)

(21) Appl. No.: 18/867,844

(22) PCT Filed: May 25, 2023
(86) PCT No.: PCT/AU2023/050443

§ 371 Date: Nov. 21, 2024

(30) Foreign Application Priority Data
May 25, 2022 (AU) 2022901415

(51) Int. Cl. A61B 5/349; A61B 5/00; A61B 5/332; G16H 50/20

(52) U.S. Cl. CPC A61B 5/349; A61B 5/332; A61B 5/7203; G16H 50/20

(57) ABSTRACT

A system and method of detecting presence of arrythmia in an electrocardiogram (ECG) signal. The method comprises steps of applying a decomposition algorithm to one or more portions of the ECG signal, each portion corresponding to at least one heartbeat. The method also comprises, for each portion, selecting at least one output of the decomposition algorithm; providing the selected at least one output to a first trained convolutional neural network (CNN) arrangement, the first CNN arrangement generating coefficients of a predetermined size; and inputting the coefficients of the predetermined size to a second trained CNN arrangement, the second CNN arrangement trained to output a classification of whether arrythmia is present in the portion of the ECG.

This application claims the benefit of priority from Australian Provisional Patent Application No. 2022901415, filed 25 May 2022, the contents of which are incorporated by reference in their entirety.

### TECHNICAL FIELD

The present invention relates generally to detecting presence of arrythmia in a cardiac signal and, in particular, to a system using convolutional neural networks to detect presence of arrythmia in a cardiac signal. The present invention also relates to a method and apparatus for detecting presence of arrythmia in a cardiac signal, and to a computer program product including a computer readable medium having recorded thereon a computer program for detecting arrythmia in a cardiac signal.

### BACKGROUND

Cardiovascular disease contributes to a high number of deaths worldwide. Cardiac arrythmia relates to an irregular rate or rhythm of a human heartbeat and is an important class of cardiovascular disease. Prompt detection of arrythmia is important in terms of cardiovascular health.

An electrocardiogram (ECG) a known method of recording operation of a human heart. An ECG measures electrical signals generated by heart activity. Recorded ECG signals are commonly used for detecting heart problems, including arrythmia.

Different types of arrythmias exist, each having characterising ECG signal patterns. In order to correctly detect cardiac arrythmia, continuous analysis of a patient's heartbeat can allow early detection of arrythmia and early treatment for cardiac health. Detecting and classifying arrhythmias can be very challenging for a medical practitioner, requiring scanning ECG data over many hours or days.

Standard medical-grade ECG measuring systems require a subject to wear typically twelve (12) electrodes connected to a monitor. While standard ECG systems are not typically prone to noise, the standard systems are not practical for long term, continuous measurements of more than 24 hours, or use where the subject is not in a single location, for example if the subject is moving, exercising, working, or undertaking everyday tasks.

Ability to monitor heart activity continuously and through a subject's day to day life may be valuable in identifying arrythmia. Wearable devices have been developed that can measure ECG signals. While wearable devices can be worn by the subject for relatively long (for example up to 7 days), continuous periods and while the subject is active, the ECG signals generated are subject to relatively high levels of noise and interference. Sources of noise and interference affecting wearable devices can include patient movement, electrode contact noise, instrumentation noise and external electromagnetic radiation.

Machine learning techniques have been developed as a method of detecting arrythmias. However, known machine learning techniques for detecting arrythmia from ECG data are very susceptible to noise in ECG readings. Existing machine learning techniques face difficulty in accurately classifying arrythmia using noisy signals such as ECG signals measured by wearable devices.

### SUMMARY

It is an object of the present invention to substantially overcome, or at least ameliorate, one or more disadvantages of existing arrangements.

One aspect of the present disclosure provides a method of detecting presence of arrythmia in an electrocardiogram (ECG) signal, the method comprising the steps of: applying a decomposition algorithm to one or more portions of the ECG signal, each portion corresponding to at least one heartbeat; for each portion: selecting at least one output of the decomposition algorithm; providing the selected at least one output to a first trained convolutional neural network (CNN) arrangement, the first CNN arrangement generating coefficients of a predetermined size; and inputting the coefficients of the predetermined size to a second trained CNN arrangement, the second CNN arrangement trained to output a classification of whether arrythmia is present in the portion of the ECG.

Another aspect of the present disclosure provides a non-transitory computer-readable storage medium storing a program for executing a method of detecting presence of arrythmia in an electrocardiogram (ECG) signal, the method comprising the steps of: applying a decomposition algorithm to one or more portions of the ECG signal, each portion corresponding to at least one heartbeat; for each portion: selecting at least one output of the decomposition algorithm; providing the selected at least one output to a first trained convolutional neural network (CNN) arrangement, the first CNN arrangement generating coefficients of a predetermined size; and inputting the coefficients of the predetermined size to a second trained CNN arrangement, the second CNN arrangement trained to output a classification of whether arrythmia is present in the portion of the ECG.

Another aspect of the present disclosure provides a system, comprising: a wearable device configured to capture an electrocardiogram (ECG) signal of a user; a memory; and a processor, wherein the processor is configured to execute code stored on the memory for implementing a method of detecting presence of arrythmia in the ECG signal; the method comprising: applying a decomposition algorithm to one or more portions of the ECG signal, each portion corresponding to at least one heartbeat; for each portion: selecting at least one output of the decomposition algorithm; providing the selected at least one output to a first trained convolutional neural network (CNN) arrangement, the first CNN arrangement generating coefficients of a predetermined size; and inputting the coefficients of the predetermined size to a second trained CNN arrangement, the second CNN arrangement trained to output a classification of whether arrythmia is present in the portion of the ECG.

Another aspect of the present disclosure provides apparatus, comprising: a memory; and a processor, wherein the processor is configured to execute code stored on the memory for implementing a method of detecting presence of arrythmia in an electrocardiogram (ECG) signal, the method comprising the steps of: applying a decomposition algorithm to one or more portions of the ECG signal, each portion corresponding to at least one heartbeat; for each portion: selecting at least one output of the decomposition algorithm; providing the selected at least one output to a first trained convolutional neural network (CNN) arrangement, the first CNN arrangement generating coefficients of a predetermined size; and inputting the coefficients of the predetermined size to a second trained CNN arrangement, the second CNN arrangement trained to output a classification of whether arrythmia is present in the portion of the ECG

Another aspect of the present disclosure provides a method of training an arrangement of convolutional neural networks (CNNs) to identify presence of arrythmia in an electrocardiogram ECG signal, the method comprising the steps of: receiving a plurality of training samples, each training sample comprising a portion of the ECG signal corresponding to at least one heartbeat and a result indicating whether arrythmia is present; applying a decomposition algorithm to each portion of the ECG; selecting a plurality of DWT coefficients for each training sample; and training the CNNs to detect presence of arrythmia by, for each training sample: providing the selected plurality of coefficients and the corresponding result to first CNN arrangement to generate coefficients of a predetermined size; and providing the generated coefficients and the corresponding result to a second CNN arrangement for classifying presence of arrythmia.

Other aspects are also disclosed.

### BRIEF DESCRIPTION OF THE DRAWINGS

At least one embodiment of the present invention will now be described with reference to the drawings, in which:

FIGS. 1A and 1B show a system for detecting presence of arrythmia in an ECG signal;

FIGS. 2A and 2B collectively form a schematic block diagram representation of an electronic device upon which described arrangements can be practised;

FIGS. 3A and 3B show example structures of a discrete waveform transform;

FIG. 4 shows an example architecture for a convolutional neural network (CNN) structure used for detecting presence of arrythmia;

FIG. 5 shows an ECG signal for a typical human heartbeat;

FIG. 6 shows a method of training the CNN architecture of FIG. 4 to detect arrythmia;

FIG. 7 shows a method of detecting presence of arrythmia in an ECG signal; and

FIGS. 8A and 8B show dataflow used in the method of detecting presence of arrythmia in an ECG signal of FIG. 7.

### DETAILED DESCRIPTION INCLUDING BEST MODE

Where reference is made in any one or more of the accompanying drawings to steps and/or features, which have the same reference numerals, those steps and/or features have for the purposes of this description the same function(s) or operation(s), unless the contrary intention appears.

It is to be noted that the discussions contained in the “Background” section and that above relating to prior art arrangements relate to discussions of documents or devices which form public knowledge through their respective publication and/or use. Such should not be interpreted as a representation by the present inventor(s) or the patent applicant that such documents or devices in any way form part of the common general knowledge in the art.

The arrangements described use signal decomposition techniques, such as a discrete wave transform (DWT), and an architecture that typically includes multiple convolutional neural networks (CNNs) to allow presence of arrythmia in electrocardiogram (ECG) signal of a patient's heartbeat to be detected. The arrangements can allow presence of arrythmia to be detected with sufficient accuracy to assist a medical practitioner even if the ECG reading was prone to noise at measurement, such as noise present in an ECG measured using a wearable device. Some embodiments described preferably use selected coefficients of a decomposed ECG signal, rather than a reconstructed decomposed signal, for improved efficiency and suitability for use on edge devices. A preferred embodiment uses two stage-processing of (i) a low dimensional discrete wavelet-based noise removal and (ii) a combination of three Convolutional Neural Networks (CNNs) for classification of arrythmia.

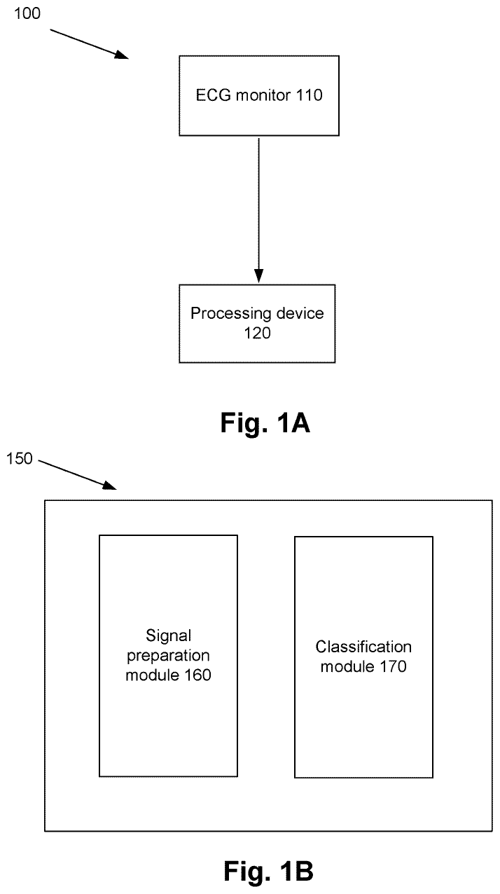

FIG. 1A shows a system 100 for detecting arrythmia in a patient's heartbeat. The system 100 includes an ECG monitor or ECG measurement device 110 and a processing device 120. The ECG measurement device 110 can be any device that is suitable for measuring an ECG signal of a human patient. In the example described herein the ECG measurement device 110 is a wearable device such that can be placed against a patient's skin to measure ECG. For example an ECG patch manufactured by IREALCARE can be used. The ECG measurement device 110 can also be a non-wearable device such as a standard, twelve-electrode ECG system used in hospitals.

The processing device 120 can be any device that is capable of receiving an ECG signal, directly or indirectly, from the ECG measurement device 110 and performing the processing described thereafter. The processing device 120 stores software capable of detecting arrythmia in the received ECG signal using the arrangements described hereafter. In some arrangements, the ECG measurement device 110 and the processing device 120 are integrated into a single device. In other arrangements, the ECG measurement device 110 and the processing device 120 are separate devices. The ECG measurement device 110 may communicate ECG signals directly to the processing device 120. Alternatively, the processing device 120 may receive the ECG signals indirectly, for example ECG signals stored on one or more external devices, such as a cloud server or a hospital server.

FIG. 1B shows a top-level software architecture 150 of software executed by the processing device 120 to determine presence of arrythmia. The architecture 150 includes a signal preparation module 160 and a classification module 170. The signal preparation module 160 receives ECG signal and prepares the ECG signals for input to the classifier module 170. The classification module 170 comprises a number of convolutional neural networks trained to classify whether the received ECG signal indicates presence of arrythmia in the patient's heart. Operation and structure of the signal preparation module 160 and the classification module 170 are discussed hereafter. In the arrangements described, the steps implemented on the processing module 120 are described in a single device. In other embodiments, the steps implemented by the processing device may be distributed or partitioned across multiple devices.

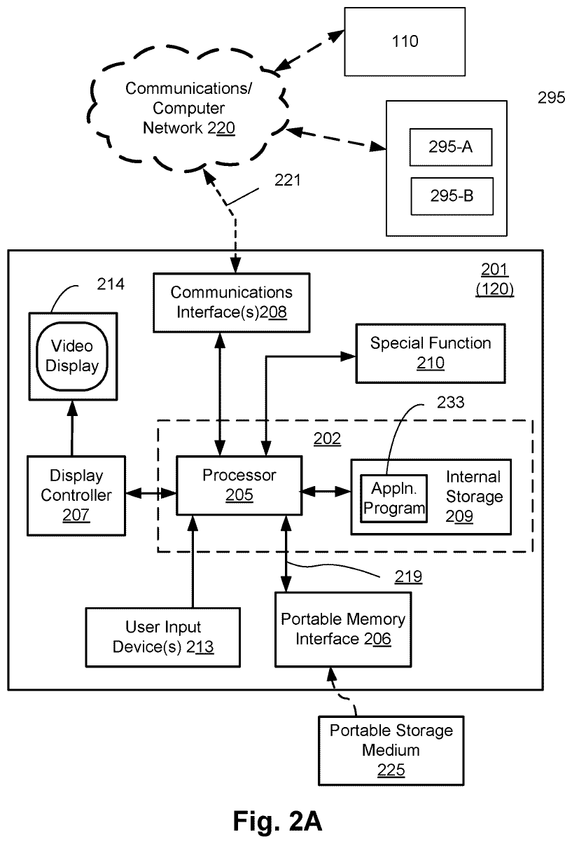

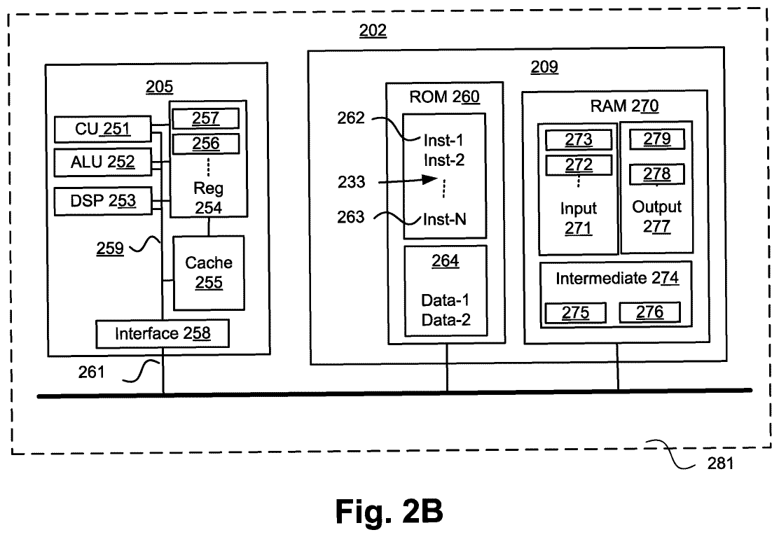

FIGS. 2A and 2B collectively form a schematic block diagram of a general purpose electronic device 201 including embedded components, upon which the methods of using an ECG to detect presence of arrythmia to be described are desirably practiced. The electronic device 201 corresponds to the processing device 120 of FIG. 1 in the examples described. The electronic device 201 may be, for example, a smartphone, a tablet or a type of edge device, in which processing resources are limited. Nevertheless, the methods to be described may also be performed on higher-level devices such as desktop computers, server computers, and other such devices with significantly larger processing resources.

As seen in FIG. 2A, the electronic device 201 comprises an embedded controller 202. Accordingly, the electronic device 201 may be referred to as an “embedded device.” In the present example, the controller 202 has a processing unit (or processor) 205 which is bi-directionally coupled to an internal storage module 209. The storage module 209 may be formed from non-volatile semiconductor read only memory (ROM) 260 and semiconductor random access memory (RAM) 270, as seen in FIG. 2B. The RAM 270 may be volatile, non-volatile or a combination of volatile and non-volatile memory.

The electronic device 201 includes a display controller 207, which is connected to a video display 214, such as a liquid crystal display (LCD) panel or the like. The display controller 207 is configured for displaying graphical images on the video display 214 in accordance with instructions received from the embedded controller 202, to which the display controller 207 is connected.

The electronic device 201 also includes user input devices 213 which are typically formed by keys, a keypad or like controls. In some implementations, the user input devices 213 may include a touch sensitive panel physically associated with the display 214 to collectively form a touch-screen. Such a touch-screen may thus operate as one form of graphical user interface (GUI) as opposed to a prompt or menu driven GUI typically used with keypad-display combinations. Other forms of user input devices may also be used, such as a microphone (not illustrated) for voice commands or a joystick/thumb wheel (not illustrated) for ease of navigation about menus.

As seen in FIG. 2A, the electronic device 201 also comprises a portable memory interface 206, which is coupled to the processor 205 via a connection 219. The portable memory interface 206 allows a complementary portable memory device 225 to be coupled to the electronic device 201 to act as a source or destination of data or to supplement the internal storage module 209. Examples of such interfaces permit coupling with portable memory devices such as Universal Serial Bus (USB) memory devices, Secure Digital (SD) cards, Personal Computer Memory Card International Association (PCMIA) cards, and the like.

The electronic device 201 also has a communications interface 208 to permit coupling of the device 201 to a computer or communications network 220 via a connection 221. The connection 221 may be wired or wireless. For example, the connection 221 may be radio frequency or optical. An example of a wired connection includes Ethernet. Further, an example of wireless connection includes Bluetooth™ type local interconnection, Wi-Fi (including protocols based on the standards of the IEEE 802.11 family), Infrared Data Association (IrDa) and the like. The electronic device can receive ECG data or signals from the ECG measurement device 110 via the network 220 for example. The electronic device 201 can receive data from other sources directly or indirectly via the network 220, for example from a server 295, for example a cloud server or a hospital server. For example, in experiments conducted two databases, indicated as databases 295-A and 295-B were accessed for training a CNN architecture to classify presence of arrythmia in an ECG signal and testing the resultant trained classifier.

Typically, the electronic device 201 is configured to perform some special function. The embedded controller 202, possibly in conjunction with further special function components 210, is provided to perform that special function. For example, the device 201 may be a mobile telephone handset. In this instance, the components 210 may represent those components required for communications in a cellular telephone environment. Where the device 201 is a portable device, the special function components 210 may represent a number of encoders and decoders of a type including Joint Photographic Experts Group (JPEG), (Moving Picture Experts Group) MPEG, MPEG-1 Audio Layer 3 (MP3), and the like.

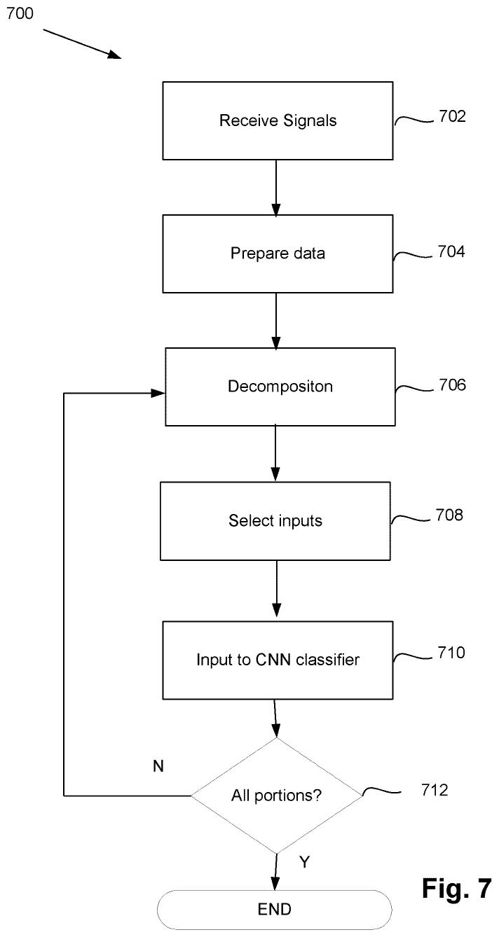

The methods described hereinafter may be implemented using the embedded controller 202, where the processes of FIGS. 6 to 8 may be implemented as one or more software application programs 233 executable within the embedded controller 202. The electronic device 201 of FIG. 2A implements the described methods. In particular, with reference to FIG. 2B, the steps of the described methods are effected by instructions in the software 233 that are carried out within the controller 202. For example, the modules 160 and 170 may be sub-modules of the software 233 stored in the memory 209. The software instructions may be formed as one or more code modules, each for performing one or more particular tasks. The software may also be divided into two separate parts, in which a first part and the corresponding code modules performs the described methods and a second part and the corresponding code modules manage a user interface between the first part and the user.

The software 233 of the embedded controller 202 is typically stored in the non-volatile ROM 260 of the internal storage module 209. The software 233 stored in the ROM 260 can be updated when required from a computer readable medium. The software 233 can be loaded into and executed by the processor 205. In some instances, the processor 205 may execute software instructions that are located in RAM 270. Software instructions may be loaded into the RAM 270 by the processor 205 initiating a copy of one or more code modules from ROM 260 into RAM 270. Alternatively, the software instructions of one or more code modules may be pre-installed in a non-volatile region of RAM 270 by a manufacturer. After one or more code modules have been located in RAM 270, the processor 205 may execute software instructions of the one or more code modules.

The application program 233 is typically pre-installed and stored in the ROM 260 by a manufacturer, prior to distribution of the electronic device 201. However, in some instances, the application programs 233 may be supplied to the user encoded on one or more CD-ROM (not shown) and read via the portable memory interface 206 of FIG. 2A prior to storage in the internal storage module 209 or in the portable memory 225. In another alternative, the software application program 233 may be read by the processor 205 from the network 220, or loaded into the controller 202 or the portable storage medium 225 from other computer readable media. Computer readable storage media refers to any non-transitory tangible storage medium that participates in providing instructions and/or data to the controller 202 for execution and/or processing. Examples of such storage media include CD-ROM, a hard disk drive, a ROM or integrated circuit, USB memory, a magneto-optical disk, flash memory, or a computer readable card such as a PCMCIA card and the like, whether or not such devices are internal or external of the device 201. Examples of transitory or non-tangible computer readable transmission media that may also participate in the provision of software, application programs, instructions and/or data to the device 201 include radio or infra-red transmission channels as well as a network connection to another computer or networked device, and the Internet or Intranets including e-mail transmissions and information recorded on Websites and the like. A computer readable medium having such software or computer program recorded on it is a computer program product.

The second part of the application programs 233 and the corresponding code modules mentioned above may be executed to implement one or more graphical user interfaces (GUIs) to be rendered or otherwise represented upon the display 214 of FIG. 2A. Through manipulation of the user input device 213 (e.g., the keypad), a user of the device 201 and the application programs 233 may manipulate the interface in a functionally adaptable manner to provide controlling commands and/or input to the applications associated with the GUI(s). Other forms of functionally adaptable user interfaces may also be implemented, such as an audio interface utilizing speech prompts output via loudspeakers (not illustrated) and user voice commands input via the microphone (not illustrated).

FIG. 2B illustrates in detail the embedded controller 202 having the processor 205 for executing the application programs 233 and the internal storage 209. The internal storage 209 comprises read only memory (ROM) 260 and random access memory (RAM) 270. The processor 205 is able to execute the application programs 233 stored in one or both of the connected memories 260 and 270. When the electronic device 201 is initially powered up, a system program resident in the ROM 260 is executed. The application program 233 permanently stored in the ROM 260 is sometimes referred to as “firmware”. Execution of the firmware by the processor 205 may fulfil various functions, including processor management, memory management, device management, storage management and user interface.

The processor 205 typically includes a number of functional modules including a control unit (CU) 251, an arithmetic logic unit (ALU) 252, a digital signal processor (DSP) 253 and a local or internal memory comprising a set of registers 254 which typically contain atomic data elements 256, 257, along with internal buffer or cache memory 255. One or more internal buses 259 interconnect these functional modules. The processor 205 typically also has one or more interfaces 258 for communicating with external devices via system bus 281, using a connection 261.

The application program 233 includes a sequence of instructions 262 though 263 that may include conditional branch and loop instructions. The program 233 may also include data, which is used in execution of the program 233. This data may be stored as part of the instruction or in a separate location 264 within the ROM 260 or RAM 270.

In general, the processor 205 is given a set of instructions, which are executed therein. This set of instructions may be organised into blocks, which perform specific tasks or handle specific events that occur in the electronic device 201. Typically, the application program 233 waits for events and subsequently executes the block of code associated with that event. Events may be triggered in response to input from a user, via the user input devices 213 of FIG. 2A, as detected by the processor 205. Events may also be triggered in response to other sensors and interfaces in the electronic device 201.

The execution of a set of the instructions may require numeric variables to be read and modified. Such numeric variables are stored in the RAM 270. The disclosed method uses input variables 271 that are stored in known locations 272, 273 in the memory 270. The input variables 271 are processed to produce output variables 277 that are stored in known locations 278, 279 in the memory 270. Intermediate variables 274 may be stored in additional memory locations in locations 275, 276 of the memory 270. Alternatively, some intermediate variables may only exist in the registers 254 of the processor 205.

The execution of a sequence of instructions is achieved in the processor 205 by repeated application of a fetch-execute cycle. The control unit 251 of the processor 205 maintains a register called the program counter, which contains the address in ROM 260 or RAM 270 of the next instruction to be executed. At the start of the fetch execute cycle, the contents of the memory address indexed by the program counter is loaded into the control unit 251. The instruction thus loaded controls the subsequent operation of the processor 205, causing for example, data to be loaded from ROM memory 260 into processor registers 254, the contents of a register to be arithmetically combined with the contents of another register, the contents of a register to be written to the location stored in another register and so on. At the end of the fetch execute cycle the program counter is updated to point to the next instruction in the system program code. Depending on the instruction just executed this may involve incrementing the address contained in the program counter or loading the program counter with a new address in order to achieve a branch operation.

Each step or sub-process in the processes of the methods (such as in FIGS. 6 and 7) described below is associated with one or more segments of the application program 233, and is performed by repeated execution of a fetch-execute cycle in the processor 205 or similar programmatic operation of other independent processor blocks in the electronic device 201.

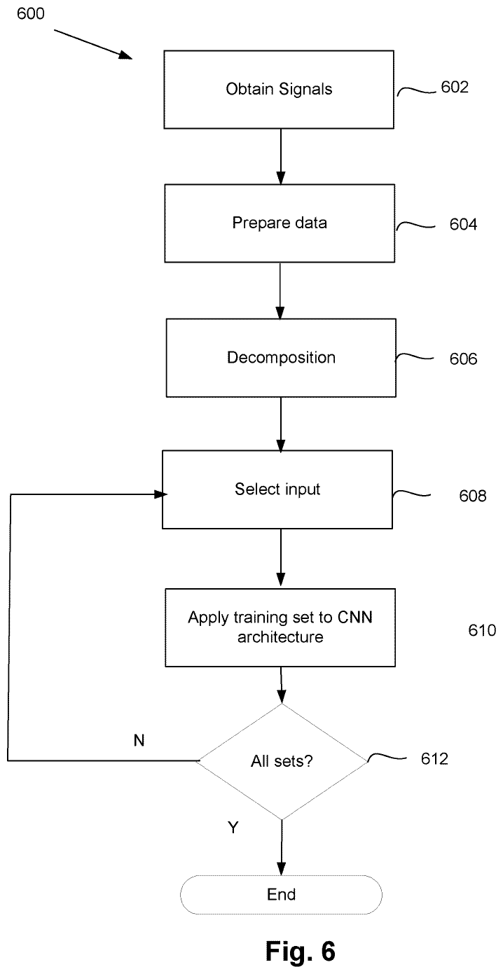

Cardiologists make arrhythmia diagnosis based on the shape and the time length of a heart-beat pulse. In ECG analysis a heartbeat can be separated into several intervals or stages. FIG. 5 shows a typical human pulse signal 500 obtained using ECG and identifies typical intervals of the pulse 500. The pulse 500 is divided into a P wave 510, a QRS complex 520, and a T wave 530. The P wave 510 has a length “PR Interval” 511 and a portion known as a “PR segment” 512. A peak R (521) has the highest amplitude in the ECG pulse 500. An R-peak forms part of the QRS waveform 520, providing a characteristic oscillation that corresponds to the contraction of the ventricles and expansion of the atria of a heart. Cardiologists make arrhythmia diagnosis based on the shape and the time length of intervals of a heart-beat pulse such as the P wave 510, QRS complex 520 and T wave 530 of the pulse 500.

The characteristic shape of an ECG heartbeat signal, as shown in FIG. 5, can be used for the labelling of data signals and the training of neural networks. In the example arrangements described, four classes of heart conditions are identified as different types of heartbeats, referred to as: (i) normal, (ii) ventricular premature beat, (iii) supraventricular premature beat, and (iv) unclassifiable beat, with the notation consistent with the Advancement of Medical Instrumentation (AAMI) EC57 standard, shown in Table 1.

TABLE 1
(AAMI) EC57 standard notation

| Heart Diseases | EC57 Standard |
|---|---|
| Normal | N |
| Ventricular premature beat | V |
| Supraventricular premature beat | S |
| Unclassifiable beat | Q |

The heart conditions identified above relate to the intervals shown for the pulse 500. For condition (i) normal, a regular PR interval (such as 511) is usually between 0.12 seconds and 0.20 seconds. The time between the Q-T period should not be greater than 0.44 seconds. The heart beats related to R-R intervals (an interval from one R-peak to a next R-peak) are usually regular for the normal condition, ranging from 60 to 100 beats per minute.

For condition (ii) ventricular premature beat, irregular heartbeats appear, leading to inequivalent R-R interval length. In addition, the QRS complex (520) of condition (ii) can be widened, often notched, and typically has a QRS duration >0.16 seconds. Condition (iv) occurs if a typical human pulse signal cannot be identified.

For condition (iii) supraventricular premature beat, irregular heartbeats appear, leading to inequivalent R-R interval length. Unlike condition (ii), the QRS duration usually has a normal time. Condition (iv) occurs if a typical human pulse signal cannot be identified.

The arrangements described were tested on two databases of ECG data. The first database was the widely used benchmark ECG dataset, MIT-BIH (corresponding to database 295-A in the example of FIG. 2A). The second database (for example relating to database 295-B in the example of FIG. 2A), was obtained from the Panjin Central Hospital, Liaoning, China. The information in database 295-A relates to ECG signals captured using standard medical ECG systems (involving 12 electrodes) that are less prone to noise but unsuitable for longer-term use. The information in database 295-B was collected using wearable devices that are prone to noise but suitable for longer-term use. The methods described were tested by identifying four classes of heart conditions, referred to as: (i) normal, (ii) ventricular premature beat, (iii) supraventricular premature beat, and (iv) unclassifiable beat, denoted by N, V, S, and Q, respectively based on the AAMI EC57 standard shown in Table 1 above. In other arrangements, training to identify other heart conditions relate to arrythmia could also be conducted.

The arrangements described process and analyse ECG signals, such as ECG signals measured from a patient for detection of arrythmia. A first stage relates to decomposing the ECG signals into frequency components. A suitable decomposition technique was found to be a Discrete Wavelet Transform (DWT). The DWT operates to both remove noise and decompose the ECG signals into different frequency bands, which are used as spectral-temporal input features of the CNN models. DWT is particularly suitable for non-stationary signals such as ECG signals. DWT operates to assist in the removal of noise from electrical signals, detection of discontinuities and the like. DWT can allow analysis in both time and frequency domains. As described below, resultant decomposed coefficients cab be used as inputs to a CNN architecture.

While DWT is particularly suitable, other techniques can use used in the decomposition stage. For example, a noise removal function or algorithm may be implemented followed by a decomposition function. Example noise removal functions may also be provided by filters, which can be classified in different ways. Filters used may be non-linear or linear, time-variant or time-invariant, discrete-time (sampled) or continuous-time, infinite impulse response or finite impulse response. Linear continuous-time filters like a Chebyshev filter and a Butterworth filter can be further classified into a low-pass filter, a high-pass filter, a bandpass filter, a band-stop filter and so on based on the frequency response of the filter. Example decomposition functions applied to the resultant noise removed signal include empirical mode decomposition, principal component analysis and the like.

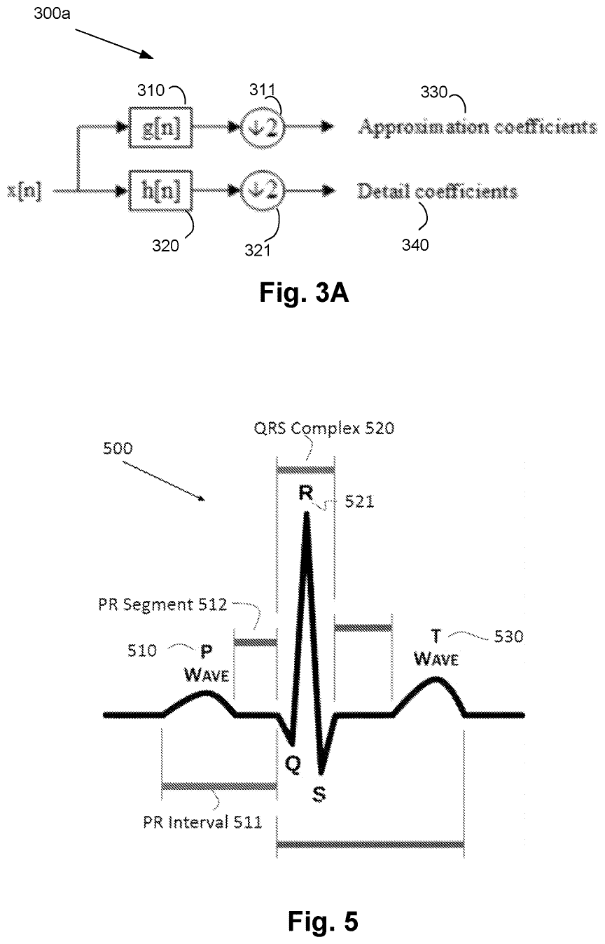

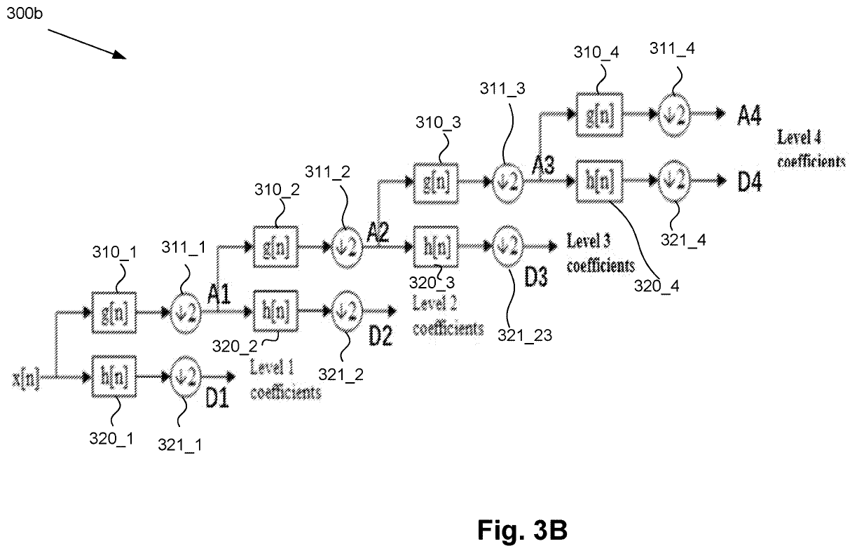

The DWT of a discrete signal x with frequency band 0-f is calculated by passing the signal through a series of low-pass and high-pass filters, followed by subsampling operators. FIG. 3A shows an architecture 300a of a single level DWT algorithm. First, a signal x[n] passes through a low-pass filter 310 and a high-pass filter 320, in parallel, with impulse responses g and h, respectively. The responses can be expressed by Equations (1) and (2) respectively:

$${y}_{1} [ n ] = ( x * g ) [ n ] = {\sum }_{k = - ∞}^{∞} x [ k ] g [ n - k ] , \qquad ( 1 )$$

$${y}_{2} [ n ] = ( x * h ) [ n ] = {\sum }_{k = - ∞}^{∞} x [ k ] h [ n - k ] , \qquad ( 2 )$$

In Equations (1) and (2), n represents the length of the discrete signal x, expressed in the number of samples. The low-pass and high-pass filters 310 and 320 are so-called “quadrature mirror filters”. The filters 310 and 320 are orthogonal to each other, and have corresponding transfer function magnitudes that are mirror images of each other around pi/2. When signal x passes through a pair of quadrature mirror filters, the signal x is projected into two orthogonal bases, where the signal projected in the low-pass filter 310 has a frequency band 0-f/2, while the signal projected in the high-pass filter 320 has the frequency band f/2-f. In other words, the signal x is decomposed by the paired quadrature mirror filters. However, the decomposing process has not finished since the signal x is only decomposed in the frequency domain but not in the time domain.

The signal x is deconstructed in the time domain using a sampling rate equal to or greater than twice the highest frequency in the signal based on the Nyquist-Shannon sampling theorem. In the example of the low-pass filter process 310, signal x can be reconstructed if the sampling rate fs is equal to or greater than 2f before the signal passes through the filter. The highest frequency in the output of the low-pass filter 310 is selected as f/2, which means that the corresponding output signal can be reconstructed if the sampling rate fs is equal to or greater than f. The halved sampling rate indicates that half of the signal samples are redundant and can be removed to reconstruct the output of the low-pas filter 310. In this way, the signal scale is doubled in the time domain. After the signal x [n] passes through the high-pass filter 320, the output frequency band lies in the range f/2-f, but no frequency band appears in the range 0-f/2. Subsampling by a factor of 2 does cause the information at frequency band f/2-f to be aliased into the information at frequency band 0-f/2. However, as there are no signal components in the range 0-f/2, the high-pass output is also subsampled at the frequency f_s=f. FIG. 3A shows subsampling modules 311 and 321 for sampling outputs of the filters 310 and 320 respectively. The outputs of the decomposition process are represented by the DWT detail and approximation coefficients.

The output of the DWT shown in the example 300 are detail coefficients 340 and approximation coefficients 330, generated by the subsampling operators 321 and 311, respectively.

In experiments conducted by the inventors, and in the example arrangements described, a DWT algorithm with at least four levels is applied to an ECG signal. The number of levels of DWT can be increased, provided that wavelet coefficients are not overly influenced by boundary effects. Boundary effects can occur if a level of decomposition is too large. If boundary effects are not observed in higher level decomposition of an ECG, the level of decomposition can be set as less than or equal to ceil(log 2(N)), where N is the length of ECG signal. The number of coefficients provided to the CNN arrangement can depend on the number of levels, the presence of noise or boundary effects, and the level of computational complexity allowable on the device 201. For example, using a set of coefficients is typically less computationally complex than using a reconstructed signal.

FIG. 3B shows an example architecture 300b of a DWT with four levels. The architecture 300b can be implemented as part of the module 160, for example. The architecture 300b has a first level having a high-pass filter 320_1 and a low-pass filter 310_1. An ECG signal x[n] is input to the filters 320_1 and 310_1. Outputs of the high-pass filter 320_1 and the low-pass filter 310_1 are input to subsampling modules or operators 321_1 and 311_1 respectively. The modules 311_1 and 321_1 generate first stage outputs, being approximation coefficients A1 and detail coefficients D1 respectively. The modules 311_1 and 321_1 typically sample at a rate of frequency f_s=f to down sample by a factor of two (half the original sampling rate). The sampling rate is selected based on sampling theory to try and avoid loss of information.

The next (second) level has a similar structure, comprising low-pass filter 310_2 and a high-pass filter 320_2. The filters 310_2 and 320_2 receive the approximation coefficients A1 as inputs. Outputs of the low-pass filter 310_2 and the high-pass filter 320_2 are input to subsampling modules 311_2 and 321_2 respectively. The modules 311_2 and 321_2 generate second stage outputs, being approximation coefficients A2 and detail coefficients D2 respectively.

The next (third) level also has a similar structure including low-pass filter 310_3 and a high-pass filter 320_3. The filters 310_3 and 320_3 receive the approximation coefficients A2 as inputs. Outputs of the low-pass filter 310_3 and the high-pass filter 320_3 are input to subsampling modules 311_3 and 321_3 respectively. The modules 311_3 and 321_3 generate third stage outputs, being approximation coefficients A3 and detail coefficients D3.

The final (fourth) level has a similar structure comprising low-pass filter 310_4 and a high-pass filter 320_4. The filters 310_4 and 320_4 receive the approximation coefficients A3 as inputs. Outputs of the low-pass filter 310_4 and the high-pass filter 320_4 are input to subsampling modules 311_4 and 321_4 respectively. The modules 311_4 and 321_4 generate fourth stage outputs, being approximation coefficients A4 and detail coefficients D4.

The filters 310_1, 310_2, 310_3 and 310_4 operate in the same manner. The filters 320_1, 320_2, 320_3 and 320_4 operate in the same manner. Similarly, the sampling modules 311_1, 311_2, 311_3, 311_4, 321_1, 321_2, 321_3, and 321_4 operate in the same manner.

In a preferred arrangement, a subset of the coefficients generated by the DWT stage are input to a plurality of CNNs. The number of coefficients depends on the level of decomposition for the first (decomposition) stage. As described above, the level of decomposition can be determined based on computational complexity and feature extraction efficiency. Using a selected subsets of the coefficients reduces the input dimensions of the proposed neural network and can decrease the computation time. Selecting coefficients rather than using a reconstructed signal makes the methods described more suitable for implementation in edge devices. The coefficients are selected based on presence of noise or information in resultant components of the decomposition algorithm. For example, in DWT, useful information is typically stored in the low-frequency bands (for example D3, D4 and A4 generated by application of the DWT 300b), while interference is involved in the high-frequency bands (for example D1 and D2 of the DWT 300b). Accordingly, the coefficients can be selected based on frequency bands where decreased noise is present. In the experiments conducted 3 coefficients were selected based on efficiency and accuracy for model training under the level of decomposition setting to 4. The coefficients D3, D4 are A4 were selected on this basis and are used in the example described. Depending on the decomposition used, the accuracy required and a level of noise expected or observed in ECG signals, different coefficients may be selected.

In other arrangements a single reconstructed signal can be generated following operation of the decomposition stage and input to a CNN arrangement. Using a single reconstructed ECG signal is less computationally efficient due to the reconstruction, and accordingly less suitable for edge devices. If different decomposition methods are used, one or more components can be selected, the number of components based upon expected noise and characteristics of the signal. For example, using empirical mode decomposition, different order components can be selected based on the orders where less noise and more useful data is present. If using principal component analysis, different order eigenvectors can be selected in a similar manner.

The decomposed signals are input to an arrangement of CNNs. Generally, a CNN consists of convolutional layers. A CNN can typically also include one or more other layers including pooling layers, dense layers and some active functions. A convolutional layer convolves the input data by using filters to reduce the input data size and extract critical data features required for further processing. A CNN can extract high-level features by feeding low-level features into multiple convolutional layers. Functionality of a pooling layer is to shrink the input signal size. Shrinking input signal size is carried out by returning either an average value (average pooling) or a maximum value (max pooling) of a typical kernel size. The kernel is a filter represented as a matrix that extracts features from the input. A dense layer (also known as a fully connected layer) is a layer with all neurons connected, and has an ability to address non-linear issues. The active functions for a convolutional and a dense layer are the Rectified Linear Unit (ReLU) and the sigmoid function, respectively. Applying a ReLU function allows and reduces likelihood of vanishing gradient, resulting in faster learning. The sigmoid function can be used after the last dense layer, which effectively maps network outputs from 0 to 1 for multiple classification values.

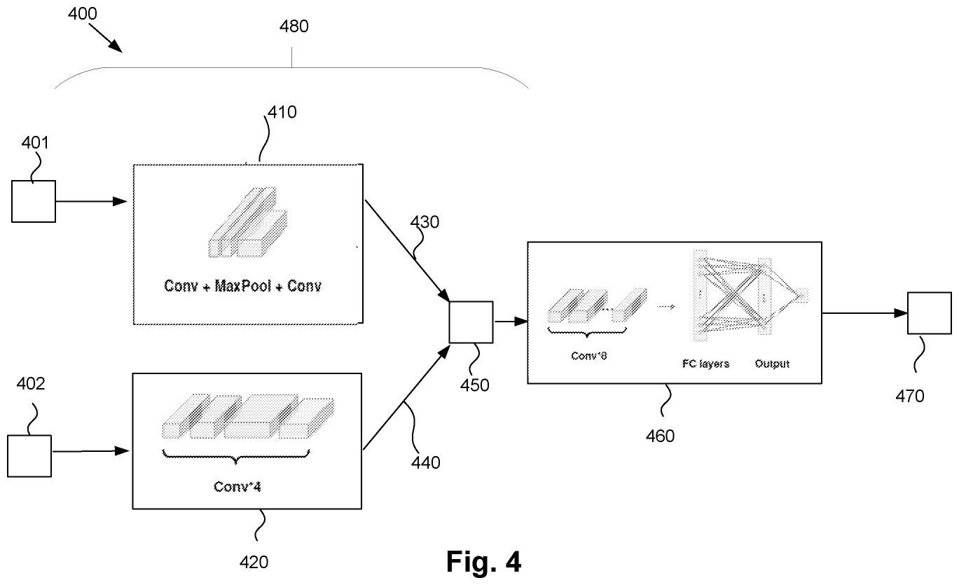

FIG. 4 shows an example architecture 400 of CNNs used in classifying presence of arrythmia from an ECG signal. In the example of FIG. 4 the architecture 400 includes two stages or arrangements, made up of three CNNs 410, 420 and 460.

The CNN 410 has a structure of a convolutional layer followed by a MaxPool layer followed by a convolutional layer in the example described. The CNN 410 receives a first input 401 from the DWT (for example 300b), corresponding to the detail coefficients D3 and generates an output 430.

The CNN 420 has a structure of four convolutional layers. The CNN receives an input 402 from the DWT (e.g. 300b) corresponding to detail coefficients D4 and approximation coefficients A4. The coefficients D4 and A4 are concatenated to form the input 402. The CNN 420 outputs coefficients 440. The output coefficients 430 and 440 have a same size or dimension. The outputs 430 and 440 can be considered to provide a set of intermediary coefficients 450. The outputs 430 and 440 are in numerical form and concatenated to provide the intermediary coefficients 450.

The structure, for example the number and type of layers, of the CNN 410 and the CNN 420 can vary based on required length of the intermediary coefficients 450. The coefficients 430 and 440 generally need to have a same length for concatenation.

The CNNs 410 and 420 in combination can be considered to provide a first CNN arrangement, referred to herein a resizing model 480. The number of CNNS used in the resizing model 480 can increase if the number of levels of the DWT architecture increases. The outputs of each CNN of the resizing model 480 is concatenated to form the intermediate coefficients 450. The resizing model 480 provides the first CNN stage of the two stages. The resizing model extracts significant signal features and adjusts the different input signal lengths to allow a specific predetermined size through the convolutional layer and pooling operations of the CNNs 410 and 420. The specific size to which the intermediate coefficients is adjusted is determined based on the required input of the classification portion of the CNN arrangement. Other variations in the resizing model can relate to the number of convolutional layers, the level of decomposition (i.e. the number of DWT filter levels), filter size and stride size for the convolutional layer, pooling operations and the input length The resized output features, the intermediate features 450, merge characteristics of inputs D3, D4 and A4. In other implementations the resizing model 480 can comprise a different number of convolutional neural networks or different structure CNNs, depending on the input size required for the CNN 460. The number of CNNS used in the resizing model can increase if the number of levels of the decomposition (DWT) architecture increases. The outputs of each CNN of the resizing model 480 is concatenated to form the intermediate coefficients 450. In implementations using different numbers of CNNs the basic resizing architecture of training inputs with different CNNs based on their input lengths, and concatenating outputs from the CNNs to the intermediate features for application to a classification CNN remains unchanged. In other arrangements, the number of CNNs in the resizing model can stay as two but the structure of the CNNs 410 and 420 can vary to provide the required output size for the intermediate coefficients 450.

In the example of FIG. 4, the resizing model comprises two CNNs (first CNN 420 and second CNN 420) and each of the selected outputs of the decomposition algorithm is provided to one of the two CNNs. In particular, detail coefficients from the third level of the DWT are input to one CNN (410), and detail and approximation coefficients from the fourth level of the DWT are input to other CNN (420). If more coefficients are selected and the number of CNNs in the resizing model changes, the coefficients will be provided to the CNNs of the resizing stage in such a manner as to ensure the intermediate coefficients are generated with the required pre-determined size.

The intermediary coefficients 450 are input to the CNN 460. The CNN 460 provides a second CNN arrangement, providing a classification stage of the architecture 400. The CNN 460 outputs a classification result 470. The classification result 470 can be one of four results: (i) normal, (ii) ventricular premature beat, (iii) supraventricular premature beat, and (iv) unclassifiable beat. The CNN arrangement 400 outputs a set of vectors, each vector corresponding to one of the heart condition outputs, for example one for each of the conditions: (i) normal, (ii) ventricular premature beat, (iii) supraventricular premature beat, and (iv) unclassifiable beat. Each output provides a value between 0 and 1 representing a probability that the corresponding condition is present. The output uses the four vectors described above to encode the classification result. For example, one hot encoding can be used whereby the outputs are reflected as: (i) normal (0001), (ii) ventricular premature beat (0010), (iii) supraventricular premature beat (0100), and (iv) unclassifiable beat (1000).

In the arrangements described the CNN 460 comprises 8 convolutional layers followed by a Fully Connected layer. In other arrangements the structure of the CNN can vary, depending on a length of the segmented signals of the ECG, the level of decomposition used in ECG, the number of selected coefficients and correspondingly the structure of the resizing model 480.

Use of first and second CNN arrangements (the resizing stage 480 and the classification stage 460) allows a number of coefficients to be used rather than a reconstructed signal. In implementations where a fully reconstructed signal is used, the resizing stage is skipped and the reconstructed signal is input directly to the classification CNN 460.

FIG. 6 shows a method 600 of training convolutional neural networks to detect presence of arrythmia. The method 600 can be implemented on the electronic device 201 using software 233 stored in the memory 209 and controlled by execution of the processor 205.

The method 600 starts at an obtaining signals step 602. The step 602 executes to obtain ECG signals. In experiments conducted in development of the invention, ECG signals were pre-recorded signals obtained from two reference databases, the databases 295-A and 295-B, for example the server 295.

A first one of the reference databases used for training, database 295-A, known as the MIT-BIH arrhythmia database, contains 48 ECG recordings by using standard 12-lead ECG sensors, each with 30 minute segments selected from 24 hrs recordings of 48 individuals. Each continuous ECG signal in the database 295-A has been passed through a bandpass filter at 0.1-100 Hz and sampled at 360 Hz. A total of 44 records from the MIT-BIH arrhythmia database are used for the performance assessment.

The second database of clinical ECG data (database 295-B) was obtained from the Panjin Central Hospital, Liaoning, China. Samples for database 295-B were collected from a wearable IREALCARE patch with a single lead. Data from 66 subjects in total was recorded. The recorded data had more than 6 million heartbeats. The signals of database 295-B were sampled at 250 Hz, and show the amplitudes of the ECG waveforms. The sample ECG data of database 295-B was more prone to noise than the samples of database 295-A.

At step 602 the processing device 120 obtains a first training sample. The training sample is selected from a set of samples from the databases 295-A and 295-B. Each training sample comprises an ECG signal and a known arrythmia result or heartbeat condition. The know result is one of (i) normal, (ii) ventricular premature beat, (iii) supraventricular premature beat, and (iv) unclassifiable beat. In the experiments conducted in development of the methods describes, 70% of samples from each of databases 295-A and 295-B were used for training. In order to allow a balanced number for different heart disease classes, the same number of ECG segments were selected for each class (for each of normal, ventricular premature beat, supraventricular premature beat and unclassifiable beat) for training.

The method 600 continues under control of the processor 205 from step 602 to a preparation step 604. The step 604 operates to prepare the training sample selected at step 602 from the databases 295-A and 295-B. The preparation involves steps such as normalisation, segmentation and splitting.

The training signals are data normalised at step 602 to change the original values in the dataset to a common scale, without distorting differences in the ranges of values or losing information. The normalising can reduce personal differences to afford weight to variable of the heartbeat signals based on general trends rather than individual anomalies. For example, z-score normalization, also known as standardization, may be used. Z-score normalization is achieved by calculate the mean μ of the data x and the data's standard deviation σ, then the normalized data xₙₒᵣₘ(=(x−μ)/σ).

The step 602 can also segment the normalised ECG samples from the databases 295-A and 295-B at the same length for neural network feeding. Step 602 effectively divides the ECG signal into one or more portions by segmenting. Each ECG segment (portion) needs to contain at least one complete heartbeat, since most existing ECG detection and classification algorithms are designed for detecting a complete heartbeat. Normal heartbeat rates vary among persons and ages, but in general, normal resting heartbeat rates are from 60 to 100 beats per minute (bpm). Considering the lowest case, i.e., 60 bpm, there is one heartbeat in one second. Therefore, to increase likelihood that at least one complete heartbeat is included in each ECG segment, the duration of the ECG segment was set to more than 1 second. The actual time interval can be calculated by dividing the segment length, expressed in the number of samples, by the sampling frequency. If the segment length is set to 610 samples for both datasets 295-A and 295-B, the time intervals can be calculated based on the given sampling frequency 360 Hz and 250 Hz for the datasets 295-A and 295-B, respectively, which are 1.70 s and 2.44 s, respectively. The calculated intervals are more than one second, which satisfies the requirement of capturing a heartbeat.

The segmentation can be varied based on the training ECG readings and their characteristics. Based on the time positions of the R peaks in the ECG waveforms, the signals are segmented (also referred to as splitting) into a fixed length with 305 portions or samples before and after the peak R. As discussed above, each segment contains 2.44 s ECG period (normally 2-3 heat beats) for the database 295-A, and 1.70 s ECG period (normally 1-1.5 heat beats) for the database 295-B. A total of 610 samples were chosen such that each segmented sample contained at least one complete heat pulse for analysis. The signals are normalized first and then segmented. Normalization is achieved by calculate the mean μ of the data x and the corresponding standard deviation σ. The normalized data is determined as x_norm(=(x−μ)/σ).

Segmentation and intervals are implemented to allow at least one heartbeat to be included in each sample provided for ECG analysis. The particular segmentation or interval length can vary to meet this requirement depending on the training data being used and variation of pulse rates therein, as well as sampling rates of the training set. The training label from the original, unsegmented ECG signal is associated with each portion or segment determined at step 602.

The method 600 continues under control of the processor 205 from step 604 to a decomposition step 606. In execution of step 606, a decomposition algorithm is applied the training samples prepared at step 604. For example, the DWT algorithm is applied based on the four layer architecture 300b shown in FIG. 3B. Alternatively, a noise removal step followed by a decomposition step can be implemented using mechanisms such as a Butterworth filter, empirical mode decomposition and the like described above. The step 606 operates to generate training set element for the classification module 170. For example, the training set element can be a subset of decomposed coefficients (for example a subset of A1 to A4 and D1 to D4 of FIG. 3B), or a reconstructed signal, or for the selected training set.

The method 600 continues under control of the processor 205 from step 606 to an input selection step 608. The step 608 executes to select a number of outputs from the step 606 to be used as inputs of the CNN architecture 400. In using the architecture 400, three sets of output coefficients are selected for each training sample, being D3, D4 and A4. The training label from the original, unsegmented ECG signal is associated with each set of coefficients selected at step 608.

The method 600 continues under control of the processor 205 from step 608 to a training step 610. At step 610 the selected coefficients and the corresponding result (presence of arrythmia type) is input to the CNN architecture 400 for training. At step 610 the selected inputs and the corresponding arrythmia result are input to the resizing stage 480 to generate intermediate coefficients 450. The intermediate coefficients 450 and the corresponding result are input to the CNN 460 to train detection of presence of arrythmia. In the experiments conducted the resizing stage 480 and the classification stage 460 were trained together the intermediate coefficients 450 being immediately treated as the input to the classification stage 460.

The method 600 continues under control of the processor 205 from step 610 to a check step 612. The step 612 operates to check if the full training set of samples has been used. If not (“N” at step 612), the method 600 returns to step 608 to select a next training sample. If all training samples have been used (“Y” at step 612), the method 600 ends. The final output of the method 600 is a trained version of the architecture 400, referred to as a trained classifier. The trained classifier can be stored in the memory 309 of the electronic device 201 for future classification, for example. The CNN arrangement was trained continuously for 100 epochs of the training data in experiments conducted.

FIG. 7 shows a method 700 of classifying presence of arrythmia from an ECG signal. The method 700 uses the classifier trained by operation of the method 600 described above, for example stored in the memory 309 of the electronic device 201. The method 700 can be implemented on the electronic device 201 using software 233 stored in the memory 209 and controlled by execution of the processor 205.

The method 700 starts at a signal receiving step 702. At step 702 the electronic device 201 receives an ECG signal. The ECG signal is typically obtained by application of the ECG measurement device 110 to a patient, for example from a wearable device worn by the patient, or from standard ECG electrodes applied to a patient. The ECG signal is received at the processing device 120 via the network 220 for example.

The method 700 continues under control of the processor 205 from step 702 to a preparation step 704. The preparation step 704 executes to prepare the ECG signal for classification. The preparation step relates to normalization and segmentation of the ECG signal received at step 702. The normalisation and segmentation are implemented at step 704 are typically implemented in the same way as the normalisation and segmentation implemented at step 602 in training the classifier architecture 400. Obtained signals re typically normalized and then segmented. Normalization is achieved by calculate the mean μ of the data x and its standard deviation σ, then the normalized data x_norm(=(x−μ)/σ). The segmentation is performed in such a way as to allow a single heartbeat to be included in each segment, as described in relation to step 604 above. The step 704 can be implemented by the module 160 for example.

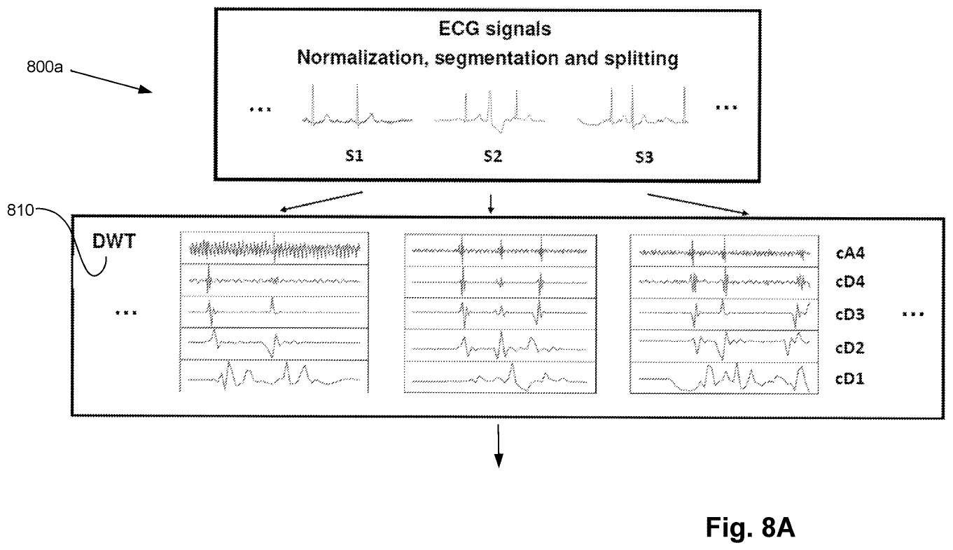

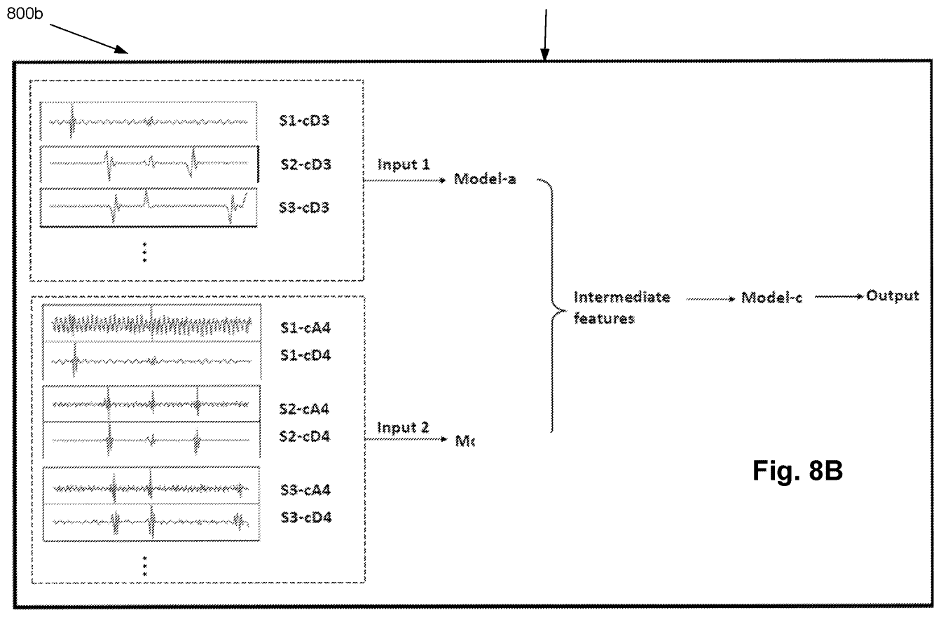

FIGS. 8A and 8B show an example dataflow of classifying presence of arrythmia in an ECG signal. FIG. 8A Shows a data flow 800a. In the data flow 800a segments S1 to S3 represent normalized, segmented results of operation of step 704 for three example ECG signals received at step 702. Each of the segments S1, S2 and S3 provides a portion of an ECG corresponding to at least a heartbeat.

The method 700 continues under control of the processor from step 704 to a decomposition step 706. At step 706 a decomposition function is applied to the each of the segments or portions prepared at step 704. For example, each portion of the ECG signal is input to a DWT function, such as the DWT architecture 300b. Alternatively, a noise removal step followed by a decomposition step can be implemented using mechanisms such as a Butterworth filter, empirical mode decomposition and the like described above. As a result of operation of step 706 one or more decomposed signals are output for each ECG segment. Using the example architecture 300b, the output signals are coefficients D1, D2, D3, A4 and D4 for each ECG segment. The step 706 can also operate to select a one or more portions of the ECG signal to be input to the next step, for example a first or next portion of the ECG signal or a first or next set of portions.

Referring to FIG. 8A, the architecture 800, the segments S1 to S3 are input to DWT 810. Coefficients cA4, cD4, cD3, cD2 and cD1 are output for each of the segments S1 to S3.

The method 700 continues under control of the processor 205 from step 706 to an input selection step 708. The step 708 executes to select inputs to be used for classification of indication of arrythmia in the ECG sign received at step 702. Preferably, inputs are selected based on reduction of a proportion of noise and to increase a proportion of ECG information in the inputs to the trained classifier. In the example described, the coefficients D3, A4 and D4 are selected. As described above, useful information is stored in the low-frequency bands (D3, D4 and A4) generated by application of the DWT 300b, while interference is involved in the high-frequency bands (D1 and D2). The number of output signals can vary based on the level of decomposition used or, in some instances, if a fully reconstructed signal is used.

FIG. 8B shows a continuation 800b of the dataflow 800a shown in FIG. 8A. The dataflow 800b shows that the following coefficients are selected for segment S1: S1-D3, S1-A4 and S1-D4. Similarly S2-D3, S2-A4 and S2-D4 are selected for segment S2 and S3-D3, S3-A4 and S3-D4 for segment S3.

The method 700 continues from step 708 to a classification step 710. The step 710 provides the inputs selected at step 708 and operates to output a classification result. The classification result is one of (i) normal, (ii) ventricular premature beat, (iii) supraventricular premature beat, and (iv) unclassifiable beat. Results (ii) ventricular premature beat, (iii) supraventricular premature beat indicate that patterns associated with arrythmia are present in the ECG signal obtained at step 702. The step 710 continues to a check step 712. The step 712 determines if all portions of the ECG signal have been classified, or if a required threshold number of portions (for example relating to a minimum required measurement time of the ECG signal) have been classified. If all or all required samples have been classified (“Y” at step 712), the method 700 ends. Otherwise (“N” at step 712) the method 700 returns to step 706 to select the next portion(s) of the ECG signal.

Each segmented portion the ECG signal generated at step corresponds to at least one heartbeat. Step 706 operates to apply the decomposition algorithm, DWT or otherwise to each portion of the ECG and step 708 to select at least one output of the decomposition algorithm. Step 710 operates to providing the selected at least one output for a segment to a first trained convolutional neural network (CNN) arrangement, being the resizing arrangement 480, and input the resultant intermediate coefficients of the predetermined size to a further trained CNN (460). The CNN 460 outputs a classification for each portion of the ECG of whether arrythmia is present in the portion of the ECG. Operation of the steps 706 to 710 to each of the segmented portions of the ECG signal can provide a single cumulative output encoded to indicate one of the designated heart conditions and thereby presence of arrythmia.

The overall output of the method 700 for all segments generated at step 704 is a result of cumulative operation of the modules 160 and 170 for a full ECG sample. In other words, the result relates to all segments in a particular ECG system being decomposed by operation of the module 160 (for example using the DWT architecture 300b) and classified by operation of the module 170 (for example using the architecture 400). As described hereinbefore, the CNN 460 generates 4 vectors which are encoded to provide a final value.

In using the example architecture of FIG. 4, the selected inputs are applied to a resizing stage 480 of the trained classifier. The selected coefficients are divided between the CNN 410 and the CNN 420. The D3 coefficient is provided to the CNN 410 and the coefficients A4 and D4 to the CNN 420.

As shown in FIG. 8B, coefficients S1-cD3 are input to Model-a, corresponding to the CNN 410. Coefficients S1-cA4 and S1-cD4 are input to Model-b, corresponding to the CNN 420. Effectively, the selected coefficients are input to the resizing stage 480 of FIG. 4. The resizing stage 480 generates intermediate coefficients 450, shown as “Intermediate features” on FIG. 8B. The intermediate coefficients 450 are input to the CNN 460 for final classification. The CNN 460 corresponds to Model-c of the dataflow 800b. The CNN 460 generates an output which encoded to indicate one of (i) normal, (ii) ventricular premature beat, (iii) supraventricular premature beat, and (iv) unclassifiable beat. Output of one of results (ii) ventricular premature beat and (iii) supraventricular premature beat indicate that patterns associated with arrythmia are present in the ECG signal obtained at step 702.

The experiments conducted used 70% of each of the databases 295-A and 295-B for training. The trained classifier was tested using the remaining 30% of samples of the databases 295-A and 295-B.

For the database 295-A, the mean testing accuracy of the experiments conducted using the architectures 300b and 400 was over 99%, where the mean accuracy is calculated by averaging the accuracy of four heartbeat types-supraventricular premature beat(S), ventricular premature beat (V), normal beat (N) and unclassifiable beat (Q), compared to the labelled signals. Since database 295-A was obtained by the standard 12-lead ECG monitors withs low interference, consideration of noise was required. The database 295-B was obtained by wearable ECG devices, and compared the mean accuracy of the proposed method with previously known CNN methods (such as described at B. Pyakillya, N. Kazachenko and N. Mikhailovsky, “Deep Learning for ECG Classification”, Journal of Physics: Conference Series, vol. 913. p. 012004, 2017) and Long short-term memory (LSTM) methods (such as those described at O. Yildirim, “A novel wavelet sequence based on deep bidirectional LSTM network model for ECG signal classification”, Elsevier, vol. 96, pp. 189 202, 2018).

The methods described were tested by identifying four classes of heart conditions, referred to as: (i) normal, (ii) ventricular premature beat, (iii) supraventricular premature beat, and (iv) unclassifiable beat, denoted by N, V, S, and Q, respectively, based on the Advancement of Medical Instrumentation (AAMI) EC57 standard. The mean obtained was 88% compared to previous methods having accuracy of 71% and 83%, respectively of the CNN and LSTM methods identified above.

The classification performance of various methods was evaluated by individual accuracy Acci and overall mean accuracy Accm, defined as shown in Equation (3) below.

$$\frac{{TP}_{i} + {TN}_{i}}{{TP}_{i} + {FP}_{i} + {TN}_{i} + {FN}_{i}} , \\ {Acc}_{m} = \frac{1}{q} {\sum }_{i = 0}^{q} {Acc}_{i} , \qquad ( 3 )$$

In Equation (3), true positive TPi is the number of correctly predicted heart conditions as positive; true negative TNi is the number of correctly predicted heart conditions as negative; false positive FPi denotes the number of incorrectly predicted heart conditions as positive; false negative FNi denotes the number of incorrectly predicted heart conditions as negative; q is the number of examined heart conditions. Acci is the measure of accuracy for each individual heart condition, which in our case were Q, N, V, and S cases. A_{ccm} is the overall mean accuracy, averaged over the four examined heart conditions.

The simulation results for identifying Q, N, V and S conditions for database 295-A had a mean accuracy of 99.1%. Table 2, compares the overall mean accuracy the A_{ccm} for the existing and the proposed methods obtained with database 295-A. The efficacy of the methods described herein is further tested with database 295-B, which contains significant interference. Simulation results for the database 295-B in the experiments conducted are shown in Tables 2 and 3, referred to as CW-CNN and CLW-CNN, respectively. ECG readings from the database 295-B were also applied to some existing CNN and LSTM techniques for comparison. The existing techniques are described in

TABLE 2
Overall accuracy of different techniques

| Year | Method | Accₘ(%) |
|---|---|---|
| 2000 | Artificial neural network with network dimension | 99.2 |
| 2009 | Extreme Learning Machine combined | 98.7 |
| | with principal component analysis | |
| 2011 | Rule-based system by taking RR intervals as inputs | 96.1 |
| 2018 | Deep bidirectional LSTM networks and | 99.4 |
| | wavelet sequences | |
| 2020 | Segmented EEG signals and novel CNN | 98.3 |
| — | CW-CNN | 99.1 |

TABLE 3
Mean accuracy of different techniques across different parameters

| Neural | | | | | |
|---|---|---|---|---|---|
| network models | Accᵢ(Q) | Accᵢ(N) | Accᵢ(V) | Accᵢ(S) | Accₘ (%) |
| Previous CNN | 0.85 | 0.89 | 0.83 | 0.28 | 71.3 |
| LSTM | 0.90 | 0.94 | 0.82 | 0.67 | 83.3 |
| CLW-CNN | 0.89 | 0.92 | 0.78 | 0.94 | 88.3 |

Table 3 indicates that the lowest overall mean accuracy of 71.3% is obtained for a CNN model described in S. L. Melo, L. P. Caloba and J. Nadal, “Arrhythmia analysis using artificial neural network and decimated electrocardiographic data,” Computers in Cardiology 2 000. Vol. 27 (Cat. 00CH37163) 00CH37163), 2000, pp. 73 76. The highest overall mean accuracy of 88.3% was achieved with the arrangements described herein. The previous CNN and LSTM models have relatively acceptable performance for classification of the type Q, N and V, but both models have low accuracies of 28% and 67%, respectively, for identifying type S. Although the previously models had relatively good performance for three types, the poor performance of identifying class S was a critical point for medical applications in practice. The methods described herein improve identifying class S to an accuracy of 94%. Although the accuracies for identifying type Q, N and V are slightly lower than for the LSTM, the arrangements described have the highest overall mean accuracy of 88%.

TABLE 4
Complexity of different models

| Neural network models | Number of trainable parameters |
|---|---|
| Previous CNN | 217,053 |
| LSTM | 7,304,581 |
| CLW-CNN | 21,720 |

In terms of complexity, the LSTM model is more computationally intensive than the methods described. The number of trainable parameters used in the methods described herein (CLW-CNN) is much lower than the number of trainable parameters in the LSTM model, as shown in Table 4. By applying the convolutional layer, the number of trainable parameters is reduced for CLW-CNN relative to the one for the LSTM, i.e. to 21,720 from 7,304,581, which indicates the complexity reduction in terms of CNN implementation.

### INDUSTRIAL APPLICABILITY

The arrangements described are applicable to the computer and data processing industries and particularly for the medical industries.

The methods described of using decomposition and a two-stage CNN architecture provide a solution that is sufficiently accurate, even in the presence of noise generated by wearables, to assist a medical practitioner in making a diagnosis of arrythmia. A patient could have readings taken over an extended period (such as 7 days) by wearing a wearable device (corresponding to 110) capable of detecting electrical heartbeat activity and generating an ECG signal. The measured ECG signal can be processed as described in relation to FIG. 7 and input to trained classifier generated according to the method 600. The extended time of wearing and the accuracy level of the result can provide a medical practitioner with a useful tool to determine whether arrythmia patterns are present in the patient's readings. The medical practitioner may also take further data into account, such as family history and any other relevant information or medical measurements, to draw a conclusion.

The arrangements described further provide a method of detecting arrythmia in a manner sufficiently computationally efficient to be implemented on an edge device. As described above, the arrangements described have a decreased number of trainable parameters compared to previous solutions but can still provide a sufficiently accurate result in a noisy signal to be useful to a medical practitioner. Selection of a number of coefficients from the DWT treated signal rather a reconstructed signal further reduces computational requirements.

The foregoing describes only some embodiments of the present invention, and modifications and/or changes can be made thereto without departing from the scope of the invention, the embodiments being illustrative and not restrictive.

(Australia Only) In the context of this specification, the word “comprising” means “including principally but not necessarily solely” or “having” or “including”, and not “consisting only of”. Variations of the word “comprising”, such as “comprise” and “comprises” have correspondingly varied meanings.

1. A method of detecting presence of arrythmia in an electrocardiogram (ECG) signal, the method comprising the steps of:
applying a decomposition algorithm to one or more portions of the ECG signal, each portion corresponding to at least one heartbeat;
for each portion:
selecting at least one output of the decomposition algorithm;
providing the selected at least one output to a first trained convolutional neural network (CNN) arrangement, the first CNN arrangement generating coefficients of a predetermined size; and
inputting the coefficients of the predetermined size to a second trained CNN arrangement, the second CNN arrangement trained to output a classification of whether arrythmia is present in the portion of the ECG.

2. The method according to claim 1, wherein the decomposition algorithm is a discrete wavelet transform (DWT).

3. The method according to claim 1, wherein the decomposition algorithm is a discrete wavelet transform (DWT) and the DWT comprises four levels and the at least one selected output comprises three sets of coefficients, the sets of coefficients selected from third and fourth levels of the DWT.

4. The method according to claim 1, wherein the first trained CNN arrangement comprises two CNNs, each of the at least one outputs of the decomposition algorithm is provided to one of the two CNNs.

5. The method according to claim 1, wherein
the first trained CNN arrangement comprises two CNNs, each of the at least one outputs of the decomposition algorithm is provided to one of the two CNNs,
the decomposition algorithm is a discrete wavelet transform (DWT), detail coefficients from a third level of the DWT are input to one of the two CNNs, and detail and approximation coefficients from a fourth level of the DWT are input to other of the two CNNs in the first trained CNN arrangement.

6. The method according to claim 1, wherein the first trained CNN arrangement comprises a first CNN and a second CNN, and an output of the first CNN is concatenated with an output of the second CNN to generate the coefficients of the predetermined size.

7. The method according to claim 1, wherein the classification indicates one of normal heartbeat, ventricular premature heartbeat, supraventricular premature heartbeat and unclassified heartbeat.

8. The method according to claim 1, wherein the first CNN arrangement comprises a first CNN and a second CNN, the first CNN having a structure of a convolutional layer followed by a MaxPool layer followed by a convolutional layer, the second CCN having a structure of four convolutional layers.

9. The method according to claim 1, wherein
the first CNN arrangement comprises a first CNN and a second CNN, the first CNN having a structure of a convolutional layer followed by a MaxPool layer followed by a convolutional layer, and the second CNN arrangement comprises a CNN having eight convolutional layers followed by a fully connected layer.

10. The method according to claim 1, further comprising apply a noise removing algorithm to the portion of the ECG signal, wherein the decomposition algorithm is applied to the portion of the ECG signal after noise removal.

11. The method according to claim 1, wherein the ECG signal is captured using a wearable device.

12. The method according to claim 1, wherein the decomposition algorithm is a discrete wavelet transform having multiple levels and the coefficients are selected based on resultant frequency bands where decreased noise is present.

13. The method according to claim 1, wherein a duration of each portion of the ECG signal is more than 1 second.

14. A non-transitory computer-readable storage medium storing a program for executing a method of detecting presence of arrythmia in an electrocardiogram (ECG) signal, the method comprising the steps of:
applying a decomposition algorithm to one or more portions of the ECG signal, each portion corresponding to at least one heartbeat;
for each portion:
selecting at least one output of the decomposition algorithm;
providing the selected at least one output to a first trained convolutional neural network (CNN) arrangement, the first CNN arrangement generating coefficients of a predetermined size; and
inputting the coefficients of the predetermined size to a second trained CNN arrangement, the second CNN arrangement trained to output a classification of whether arrythmia is present in the portion of the ECG.

15. (canceled)

16. Apparatus, comprising:
a memory; and
a processor, wherein the processor is configured to execute code stored on the memory for implementing a method of detecting presence of arrythmia in an electrocardiogram (ECG) signal, the method comprising the steps of:
applying a decomposition algorithm to one or more portions of the ECG signal, each portion corresponding to at least one heartbeat;
for each portion:
selecting at least one output of the decomposition algorithm;
providing the selected at least one output to a first trained convolutional neural network (CNN) arrangement, the first CNN arrangement generating coefficients of a predetermined size; and
inputting the coefficients of the predetermined size to a second trained CNN arrangement, the second CNN arrangement trained to output a classification of whether arrythmia is present in the portion of the ECG

17. (canceled)
